(19) United States
(12) Patent Application Publication
(10) Pub. No.: US 2025/0002625 A1
(43) Pub. Date: Jan. 2, 2025

(54) THICKENING POLYMERIC COMPOSITION FOR COSMETIC AND DETERGENT COMPOSITIONS

(71) Applicant: SNF GROUP, ANDREZIEUX-BOUTHEON (FR)

(72) Inventors: Frédéric BLONDEL, ANDREZIEUX-BOUTHEON (FR); Céline BESSET, ANDREZIEUX-BOUTHEON (FR); Benoît CLEMENT, ANDREZIEUX-BOUTHEON (FR)

(21) Appl. No.: 18/695,812

(22) PCT Filed: Oct. 5, 2022
(86) PCT No.: PCT/EP2022/077691

§ 371 Date: Mar. 26, 2024

(30) Foreign Application Priority Data
Oct. 5, 2021 (FR) FR2110531

(51) Int. Cl. C08F 220/34; A61K 8/81; A61Q 19/00; C11D 3/00; C11D 3/37

(52) U.S. Cl. CPC C08F 220/34; A61K 8/817; A61Q 19/00; C11D 3/0005; C11D 3/3769; A61K 2800/48; A61K 2800/5424

(57) ABSTRACT

A polymer composition for thickening a cosmetic or detergent composition, the polymer composition including either at least one polymer cross-linked with at least one labile cross-linker and at least one non-labile cross-linker, or at least two cross-linked polymers, at least one being cross-linked with at least one labile cross-linker and at least one being cross-linked with at least one non-labile cross-linker. The polymer composition makes it possible to maintain the viscosity of a cosmetic or detergent composition over time.

### CROSS-REFERENCE TO RELATED APPLICATIONS

This application claims benefit under 35 USC § 371 of PCT Application No. PCT/EP2022/077691 entitled THICKENING POLYMERIC COMPOSITION FOR COSMETIC AND DETERGENT COMPOSITIONS, filed on Oct. 5, 2022 by inventors Frédéric Blondel, Céline Besset and Benoît Clement. PCT Application No. PCT/EP2022/075444 claims priority of French Patent Application No. 21 10531, filed on Oct. 5, 2021.

### TECHNICAL FIELD OF THE INVENTION

The invention relates to a polymeric composition for thickening a cosmetic or detergent composition.

### BACKGROUND OF THE INVENTION

Cosmetic and detergent compositions, such as fabric softeners, present stability problems during their shelf life and/or storage by the consumer. These instabilities are correlated with an increase in the electrolyte content of the compositions over time. These electrolytes are derived from secondary reactions of ingredients in the composition, such as quaternary amine esters. Time and temperature are the main parameters influencing these secondary reactions.

Thickening polymers, obtained by inverse emulsion, such as quaternised dimethylaminoethyl (meth)acrylate ((M)ADAME)-based polymers, are known for stabilising cosmetic and detergent compositions. The stabilising property is provided by a compact stack of hydrated polymers (or hydrogels) thickening the aqueous phase of the cosmetic or detergent composition by a charge repulsion mechanism. The range of viscosity obtained depends on the volume developed by the hydrogel, which in turn depends on the structure of the polymer and more precisely the cross-linking density in the network of the polymer of which it is made.

The viscosity obtained by this mechanism is more sensitive to the presence of electrolytes in the stabilised medium. The quantity of electrolytes generated by the secondary reactions mentioned above leads to a drop in viscosity and therefore in the stability of cosmetic and detergent compositions over time.

Electrolytes are positively or negatively charged chemical substances capable of carrying or conducting an electrical charge, usually in a solution.

Currently, to compensate for this loss of viscosity or stability, manufacturers overdose cosmetic and detergent compositions with thickening polymers. This solution is of course unsatisfactory and the plaintiff has sought a technical alternative.

### SUMMARY OF THE DESCRIPTION

The problem which the invention sets out to solve is to provide an effective thickener for cosmetic and detergent compositions, making it possible to compensate for the loss of viscosity over time of the said cosmetic and detergent compositions.

The invention relates to a polymeric composition for thickening cosmetic and detergent compositions which makes it possible to significantly reduce the loss of viscosity over time of the said compositions, and preferably to maintain their viscosity over time.

The improved performance achieved by the invention makes it possible to reduce the quantity of thickening polymer, which means a reduction in greenhouse gas emissions such as CO₂ associated with the manufacture and use of these polymers.

### DETAILED DESCRIPTION

The invention relates to a polymeric composition, for thickening a cosmetic or detergent composition, said polymeric composition comprising:
Or at least one polymer cross-linked with at least one labile cross-linker and at least one non-labile cross-linker,
Or at least two cross-linked polymers, at least one being cross-linked with at least one labile cross-linker, and at least one being cross-linked with at least one non-labile cross-linker.

The invention also relates to a cosmetic or detergent composition comprising at least one thickening polymer composition according to the invention.

The invention also relates to a method for viscosifying a cosmetic or detergent composition with at least one thickening polymer composition according to the invention.

Finally, the invention concerns a method for maintaining the viscosity of a cosmetic or detergent composition over time with at least one thickening polymer composition according to the invention.

The polymer composition according to the invention enables a constant viscosity to be maintained over time despite an increase in the concentration of electrolyte in the cosmetic or detergent composition. This eliminates the need to overdose the thickening polymer.

The expression “maintaining viscosity over time” means that the viscosity of the cosmetic or detergent composition thickened with the polymeric composition according to the invention is maintained at the same value over a period of at least one year, preferably at least three years. This expression also includes the case where the viscosity is slightly reduced (by a maximum of 15% of the initial value, preferably by a maximum of 10%, more preferably by a maximum of 5%), but also the case where the loss of viscosity is significantly reduced (by at least 50%, preferably by at least 80%) compared with a cosmetic or detergent composition not comprising the polymeric composition according to the invention, after a period of one year.

Without wishing to be bound by any theory, the addition of a labile cross-linker in combination with a non-labile cross-linker compensates for the drop in viscosity associated with the increase in electrolyte concentration in the medium. When the polymer composition is added to the cosmetic or detergent composition, a compact stack of hydrated polymers, also known as hydrogels, is formed, as mentioned above. Hydrolysis of the labile cross-linker (de-cross-linking) over time and under the action of temperature increases the volume of the hydrogels in the cosmetic or detergent composition and thus maintains the volume fraction occupied in the said cosmetic or detergent composition. The viscosity of the cosmetic or detergent composition is therefore maintained over time.

The viscosity is measured with a Brookfield device, the measurement being carried out at 20° C. Those skilled in the art will know how to adapt not only the modulus of the Brookfield viscometer but also the speed of the modulus according to the viscosity of the fluid whose viscosity they wish to measure. In the following, this viscosity is referred to as Brookfield viscosity or simply viscosity.

In a first alternative, the polymeric composition according to the invention comprises at least one polymer cross-linked with at least one labile cross-linker and at least one non-labile cross-linker. Preferably, said polymer is cross-linked with a single labile cross-linker and with a single non-labile cross-linker. It can, of course, be cross-linked with several labile cross-linkers and several non-labile cross-linkers.

In a second alternative, the polymeric composition according to the invention comprises at least one polymer cross-linked with at least one labile cross-linker and at least one polymer cross-linked with at least one non-labile cross-linker.

The cross-linked polymer according to the invention is preferably a synthetic polymer. It is preferably obtained from at least one monomer with ethylenic unsaturation. It is preferably a polymer obtained from at least one non-ionic monomer and/or at least one anionic monomer and/or at least one cationic monomer and/or a zwitterionic monomer.

Labile cross-linker means a cross-linker that can be degraded by certain heat conditions and/or over time, after it has been incorporated into the polymer structure, to reduce the degree of cross-linking in the cross-linked polymer. Storage conditions, temperature and the presence of electrolytes cause bond cleavage in the labile cross-linker without substantially degrading the rest of the polymer backbone.

More specifically, in this invention, labile cross-linker is taken to mean a cross-linker of which at least 10 mol % is degraded in 5 to 10 days, at a temperature of between 5° and 100° C.

The degree of degradation of the labile cross-linker can be determined by comparative measurement against standards. The measurement is made in a 1% by weight solution of polymer containing the labile cross-linker, at pH 2.5.

The labile cross-linker may be selected from diacrylamides and methacrylamides of diamines such as piperazine diacrylamide, di, tri, tetra esters of acrylate or methacrylate such as ethylene glycol diacrylate, polyethylene glycol diacrylate, trimethylopropane trimethacrylate, ethoxylated trimethylol triacrylate, ethoxylated pentaerythritol tetracrylate, and vinyl or allyl esters of di- or tri-functional acids.

The labile cross-linking agent may also be selected from diamine diacrylamides and methacrylamides such as piperazine diacrylamide, acrylate or methacrylate esters, ethylene glycol diacrylate polyethylene glycol diacrylate, trimethylopropane trimethacrylate, ethoxylated trimethylol triacrylate, ethoxylated pentaerythritol tetracrylate, and vinyl or allyl esters of di- or tri-functional acids. The labile cross-linker is preferably chosen from water-soluble diacrylates such as PEG 200 diacrylate, PEG 400 diacrylate, PEG 600 diacrylate, PEG 1000 diacrylate, ethylene glycol diacrylate, diethylene glycol diacrylate, trethylene glycol diacrylate, tetraethylene glycol diacrylate, and polyfunctional vinyl derivatives of a polyhydric alcohol such as ethoxylated (9-20) trimethylol triacrylate.

Acrylate or methacrylate di, tri or tetra esters are preferably obtained by esterification between a compound comprising at least two OH functions, preferably two, three or four OH functions, and acrylic or methacrylic acid.

The amount of labile cross-linker in the cross-linked polymer is preferably between 100 and 200,000 ppm, more preferably between 200 and 100,000 ppm, even more preferably between 300 and 70,000 ppm, even more preferably between 400 and 50,000 ppm, even more preferably between 500 and 30,000 ppm on a total monomer basis. The ppm are expressed by weight.

Non-labile cross-linker means a cross-linker that is not degraded under temperature conditions or over time. A non-labile cross-linker is used to control the expanded conformation of the hydrogel. In particular, a non-labile cross-linker is different from a labile cross-linker as defined above, i.e. less than 1 mol % is degraded in 5 to 10 days, at a temperature of between 5° and 100° C.

The non-labile cross-linker can be selected from methylene bisacrylamide, diallylamine, triallylamine, divinyl sulphone, diethylene glycol diallyl ether and the like. The non-labile cross-linker is preferably methylene bisacrylamide.

The amount of non-labile cross-linker in the cross-linked polymer is preferably between 1 and 2000 ppm, more preferably between 10 and 1500 ppm, and even more preferably between 20 and 1000 ppm, and even more preferably between 100 and 1000 ppm based on total monomer. The ppm are expressed by weight.

The non-ionic monomer or monomers which can be used in the context of the invention can be chosen, in particular, from the group comprising water-soluble vinyl monomers. Preferred monomers belonging to this class are, for example, acrylamide, methacrylamide, N-isopropylacrylamide, N,N-dimethylacrylamide and N-methylolacrylamide. N-vinylformamide, N-vinylacetamide, N-vinylpyridine and N-vinylpyrrolidone, acryloyl morpholine (ACMO), glycidyl methacrylate, glyceryl methacrylate and diacetone acrylamide can also be used. A preferred non-ionic monomer is acrylamide.

The anionic monomer or monomers are preferably chosen from acrylic acid, methacrylic acid, itaconic acid, maleic acid, acrylamido tertiary butyl sulphonic acid (also called ATBS or 2-acrylamido-2-methylpropane sulphonic acid), vinylsulphonic acid, vinylphosphonic acid, the said anionic monomer being unsalified, partially or totally salified, and salts of 3-sulphopropyl methacrylate. The salified form advantageously corresponds to salts of alkali metals (Li, Na, K . . . ), alkaline earth metals (Ca, Mg . . . ) or ammonium, in particular quaternary ammoniums. Preferred anionic monomers are ATBS and acrylic acid or their salts.

Above and below, cationic monomers and anionic monomers, such as MADAME and ATBS, include non-salified, salified, partially salified and fully salified forms.

The cationic monomer or monomers that can be used in the context of the invention can be chosen, in particular, from monomers of the acrylamide, acrylic, vinyl, allylic or maleic type possessing a quaternary ammonium function by salification or quaternisation. These include, but are not limited to, quaternised dimethylaminoethyl acrylate (ADAME), quaternised dimethylaminoethyl methacrylate (MADAME), dimethyldiallylammonium chloride (DADMAC), acrylamido propyltrimethyl ammonium chloride (APTAC) and methacrylamido propyltrimethyl ammonium chloride (MAPTAC). The preferred cationic monomers are quaternised dimethylaminoethyl acrylate (ADAME) and quaternised dimethylaminoethyl methacrylate (MADAME), quaternisation preferably being done with the chloride ion.

Zwitterionic monomer(s) can also be used in the context of the invention; they combine both anionic and cationic charges on a single monomer. They can be chosen in particular from monomers of the betaine, sultaine, sulphobetaine, phosphobetaine and carboxybetaine types. Examples of zwitterionic monomers include sulphopropyl dimethylammonium ethyl methacrylate, sulphopropyl dimethylammonium propylmethacrylamide, sulphopropyl 2-vinylpyridinium and phosphato ethyl trimethylammonium ethyl methacrylate.

The cross-linked polymer according to the invention is preferably a cationic or anionic polymer.

When the cross-linked polymer is cationic, it is preferably either a cationic homopolymer or a copolymer comprising at least one cationic monomer and at least one non-ionic monomer. In this case, the amount of cationic monomer is preferably between 50 and 100 mol %, more preferably between 70 and 90 mol % and the amount of non-ionic monomer is preferably between 0 and 50 mol %, more preferably between 10 and 30 mol %.

When the cross-linked polymer is anionic, it is preferably either an anionic homopolymer or a copolymer comprising at least one anionic monomer and at least one non-ionic monomer. In this case, the amount of anionic monomer is preferably between 50 and 100 mol %, and the amount of non-ionic monomer is preferably between 0 and 50 mol %.

In the second alternative, the two polymers preferably have the same ionicity. In other words, if the polymer cross-linked with at least one labile cross-linker is cationic, then the polymer cross-linked with a non-labile cross-linker is also cationic.

Generally speaking, the cross-linked polymer produced according to the invention does not require the development of any particular polymerisation process. Indeed, it can be obtained by all polymerisation techniques well known to the person skilled in the art. These may include solution polymerisation; gel polymerisation; precipitation polymerisation; emulsion polymerisation (aqueous or inverse); suspension polymerisation; reactive extrusion polymerisation; water-in-water polymerisation; or micellar polymerisation.

The polymer according to the invention is preferably obtained by inverse emulsion polymerisation. The inverse emulsion is preferably distilled to obtain a polymer concentration of preferably between 3 and 65% by weight.

In the second alternative, the two polymers are preferably prepared separately from each other and then mixed to obtain the thickening polymer composition of the invention. The weight ratio of one to the other is preferably between 10:90 and 90:10. When the polymers are prepared separately by inverse emulsion, it is preferable to mix the two inverse emulsions in the appropriate proportions and then distil the resulting mixture to obtain a polymer concentration of preferably between 3 and 65% by weight.

Preferably, the polymer composition of the invention is an inverse emulsion, in which case the polymer is in the water droplets of the emulsion.

Polymerisation is generally free radical polymerisation. Free radical polymerisation includes free radical polymerisation using UV, azo, redox or thermal initiators, as well as controlled radical polymerisation (CRP) or matrix polymerisation techniques.

The thickening polymer composition can be used according to the knowledge and practices of cosmetic and detergent composition formulators.

The invention also relates to a cosmetic or detergent composition comprising at least one thickening polymer composition according to the invention.

In the present invention, cosmetic compositions also include dermatological and pharmaceutical compositions. They generally include an aqueous phase. They are applied in particular to the skin or hair and are generally in the form of oil-in-water emulsions, and sometimes water-in-oil emulsions, to form creams or lotions, for example. Such compositions may, for example, be anti-ageing creams or lotions, aftershaves, moisturisers, hair colouring lotions, shampoos, conditioners, shower products, cleansing creams, ointments or sun creams or lotions. Creams are distinguished from lotions by their higher viscosity.

Thickeners or rheology modifiers are widely used in these compositions to adapt their sensory profile (appearance, application) to consumer requirements, but also to suspend or stabilise active ingredients.

By detergent compositions we mean compositions for cleaning various surfaces, in particular textile fibres, hard surfaces of all kinds such as crockery, floors, glass, wood, metal or composite surfaces. Such compositions include, for example, detergents for washing clothes manually or in a washing machine, products for cleaning dishes manually or for dishwashers, detergent products for washing household interiors such as kitchen units, toilets, furnishings, floors, windows, and other general-purpose cleaning products.

The amount of the thickening polymer composition according to the invention added to the cosmetic or detergent composition is preferably between 0.01 and 5% by weight, preferably between 0.1 and 3% by weight.

In the first alternative according to the invention, the quantity of cross-linked polymer, with at least one labile cross-linker and at least one non-labile cross-linker, added to the cosmetic or detergent composition is preferably between 0.005 and 3% by weight, more preferably between 0.05 and 2% by weight.

In the second alternative according to the invention, the amount of polymer cross-linked with at least one labile cross-linker added to the cosmetic or detergent composition is preferably between 0.0005 and 2.7% by weight, more preferably between 0,005 and 1.8% by weight, and the amount of polymer cross-linked with at least one non-labile cross-linker added to the cosmetic or detergent composition is preferably between 0.0005 and 2.7% by weight, more preferably between 0.005 and 1.8% by weight.

Cosmetic and detergent compositions also contain other ingredients that are well known to the skilled person, such as water, mineral or vegetable oils, organic solvents, surfactants, cosmetic or pharmaceutical active ingredients, vitamins, chelating agents, emollients, moisturising agents, botanical extracts, sunscreens, detergency or cleaning adjuvants, fluorescent agents, foaming agents or foam suppressants, neutralising agents, antiseptic agents, etc, botanical extracts, sunscreens, detergent or cleaning adjuvants, fluorescent agents, foaming agents or foam suppressants, neutralising agents, pH adjusting agents, preservatives, perfumes, opacifying compounds, colouring agents, without this list being exhaustive. Those skilled in the art will know how and with which ingredients to formulate a cosmetic or detergent composition. For cosmetic compositions, reference may be made to application FR 2 979 821, and for detergent compositions reference may be made to documents FR 2 766 838, FR 2 744 131, EP 0 759 966.

The invention also relates to a method of viscosifying a cosmetic or detergent composition with at least one thickening polymeric composition as previously described. The method comprises using the polymeric composition according to the invention by techniques known to the person skilled in the art. The method therefore comprises a step of adding the polymeric composition according to the invention to the said cosmetic or detergent composition. The quantities of polymeric composition are as described above.

The invention also concerns the use of at least one polymeric composition according to the invention to viscositfy a cosmetic or detergent composition. The quantities of polymeric composition are as described above.

The invention relates to a method of maintaining the viscosity of a cosmetic or detergent composition over time with at least one thickening polymeric composition as previously described. The method therefore comprises a step of adding the polymeric composition according to the invention to the said cosmetic or detergent composition. The quantities of polymeric composition are as described above.

Finally, the invention concerns the use of at least one polymeric composition according to the invention to maintain the viscosity of a cosmetic or detergent composition over time. The quantities of polymeric composition are as described above.

### BRIEF DESCRIPTION OF THE DRAWINGS

FIG. 1 is a schematic illustration of the phenomena of loss of viscosity over time according to the prior art (upper part), and of the strategy for maintaining viscosity over time using the polymer composition according to the invention.

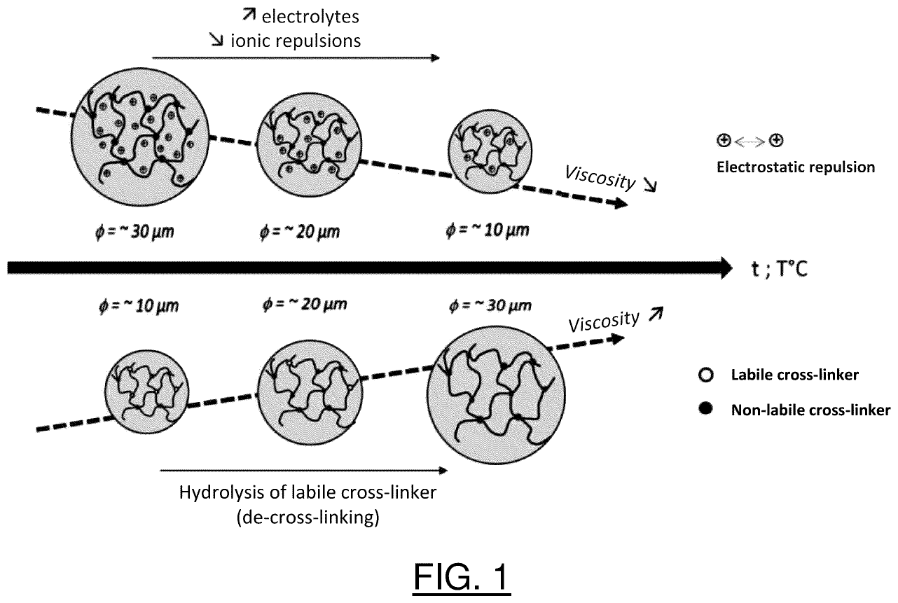

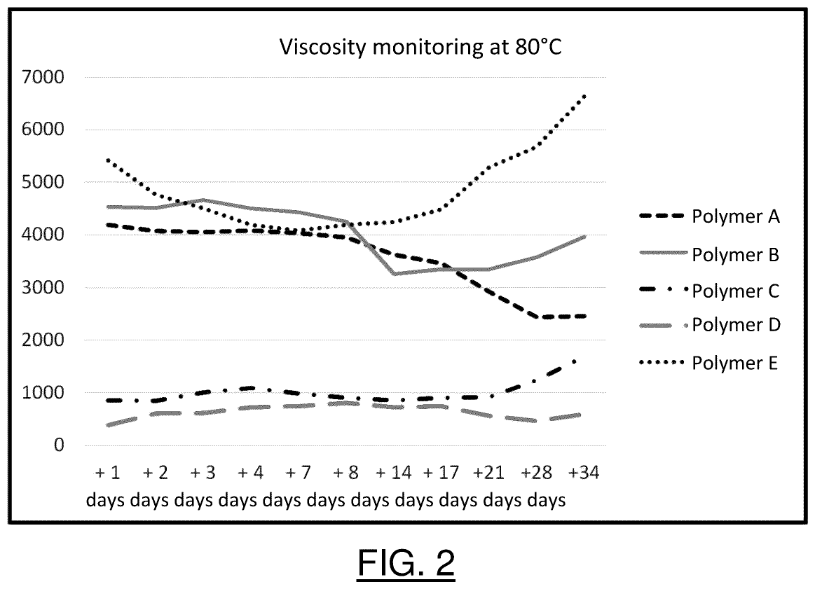

In the upper part of FIG. 1, a hydrogel obtained from a non-labile cross-linking agent initially has a diameter of 30 μm, then with time, the action of electrolytes and temperature, its diameter gradually decreases to 20 μm then 10 μm resulting in a drop in viscosity of the thickened medium. The increasing presence of electrolytes reduces the ionic repulsions responsible for the viscosifying effect leading to this loss of viscosity.

In the lower part of FIG. 1, a hydrogel of the polymer obtained from a non-labile cross-linker and a labile cross-linker initially has a size of 10 μm hydrolysis of the labile cross-linker (de-cross-linking), its size gradually increases to 20 μm, then to 30 μm, leading to an increase in the viscosity of the thickened medium over time. This increase in viscosity compensates over time for the loss of viscosity observed in the upper part of FIG. 1.

FIG. 2 is a graph representing the evolution of the viscosity of the polymer solution A to E at a concentration of 1% at 80° C. in order to simulate the action of electrolytes that form during the storage of a cosmetic or detergent composition.

### EXAMPLES

All quantities expressed in ppm are relative to the total quantity of monomer by weight.

### Composition 1 (Comparative): Synthesis of Cationic Polymer by Inverse Emulsion with Non-Labile Cross-Linker (Composition A)

The aqueous phase is prepared in a 1 L beaker by mixing together the following ingredients:
470.0 g of a solution of chlorinated dimethylaminoethyl acrylate (75% by weight in water),
60.0 g of an acrylamide solution (50% by weight in water),
0.19 g methylenebisacrylamide (541 ppm),
The pH is adjusted to 5.2+/−0.1 with a citric acid solution.

The organic phase is prepared in a 1 L glass reactor with stirring by mixing together the following ingredients:
20.0 g sorbitan monooleate,
25.0 g polymeric stabiliser (Hypermer 6212)
210.0 g white mineral oil,
63.0 g aliphatic hydrocarbon (Isopar L)

The aqueous phase is gradually transferred to the organic phase with moderate mechanical agitation. The pre-emulsion thus formed is then subjected to high shear for 1 minute (Ultra Turax, IKA).

The inverse emulsion is then degassed for 30 minutes using a nitrogen bubbling system. Polymerisation is carried out by adding a 1% by weight aqueous solution of a redox couple of sodium metabisulphite and tert-butylhydroperoxide at a flow rate of 10 ml/h.

Once the maximum temperature has been reached (adiabatic polymerisation), the temperature of the reaction medium is maintained for 60 minutes before cooling.

The emulsion obtained is then distilled under reduced pressure to remove the water and volatile organic solvent, giving a product with 58% by weight polymeric active ingredient after distillation.

Finally, in the last stage, 50.0 g of an oil-in-water emulsifier of the 6-mol ethoxylated tridecyl alcohol type is added to obtain the ready-to-use liquid polymer dispersion.

### Example 2: Synthesis of Cationic Polymer by Inverse Emulsion with Labile and Non-Labile Cross-Linker (1100 ppm Labile Cross-Linker, Composition B)

The protocol of Example 1 is reproduced by adding 0.42 g (1100 ppm) of labile cross-linker polyethylene glycol (200) diacrylate (PEG200DA, Sartomer SR 259).

### Example 3: Synthesis of Cationic Polymer by Inverse Emulsion with Labile and Non-Labile Cross-Linker (3500 ppm Labile Cross-Linker, Composition C)

The protocol in Example 2 is reproduced by changing the amount of labile cross-linker to 1.33 g (3500 ppm/active ingredient).

### Example 4: Synthesis of Cationic Polymer by Inverse Emulsion with Labile and Non-Labile Cross-Linker (5000 ppm Labile Cross-Linker, Composition D)

The protocol in Example 2 is reproduced by changing the amount of labile cross-linker to 1.91 g (5000 ppm/active ingredient).

### Example 4: Synthesis of Cationic Polymer by Inverse Emulsion with Labile Cross-Linker (4000 ppm, Composition D′)

The protocol of Example 2 is reproduced by changing the amount of labile cross-linker to 1.91 g (5000 ppm) and the amount of non-labile cross-linker to 0 g.

### Example 5: Combination of a Polymer Comprising a Non-Labile Cross-Linker and a Polymer Comprising a Labile Cross-Linker (Composition E)

Composition E is obtained by mixing 50/50% by weight of Composition A (non-labile cross-linker) with Composition D′ (labile cross-linker) before their respective distillation stages, then distilling the mixture of the two compositions.

### Example 6: Study of Compositions A to E

### Preparation of Polymer Solutions and Measurement of Viscosity

A solution of each Composition A to E is prepared to 1.0% commercial emulsion by adding 5.0 g of the corresponding liquid dispersion obtained according to examples 1 to 5 in 495.0 g of deionised water acidified to pH=2.8+/−0.1 (citric acid) under half-moon mechanical stirring at 600 RPM for 15 min.

Viscosity is measured on a Brookfield RVT viscometer at a shear rate of 10 RPM.

### Viscosity Profile Monitoring

The solutions of each Composition A to E are placed in an oven at 80° C. to simulate the accelerated ageing of the polymer over time and the impact of the electrolytes that form when a detergent or cosmetic composition is stored.

Brookfield viscosities are monitored over time at a temperature of 23° C.+/−1° C. Viscosity is measured over several days.

The results of the viscosity profile monitoring are summarised in Table 1.

TABLE 1

| | Viscosity (RVT V10) | | | | | | | | | | | |
|---|---|---|---|---|---|---|---|---|---|---|---|---|
| Composition | T₀ | +1 d | +2 d | +3 d | +4 d | +7 d | +8 d | +14 d | +17 d | +21 d | +28 d | +34 d |
| Composition | 4010 | 4190 | 4070 | 4050 | 4080 | 4030 | 3950 | 3620 | 3460 | 2920 | 2440 | 2460 |
| A | | | | | | | | | | | | |
| Drop/Gain in | | 4% | −3% | −3% | −3% | −4% | −6% | −14% | −17% | −30% | −42% | −37% |
| viscosity | | | | | | | | | | | | |
| Composition | 4300 | 4530 | 4510 | 4660 | 4500 | 4430 | 4250 | 3260 | 3350 | 3350 | 3570 | 3960 |
| B | | | | | | | | | | | | |
| Drop/Gain in | | 5% | −0.4% | 2.9% | −0.7% | −2.2% | −6.2% | −28.0% | −26.0% | −26.0% | −21.2% | −8% |
| viscosity | | | | | | | | | | | | |
| Composition | 500 | 852 | 844 | 1000 | 1090 | 980 | 900 | 850 | 900 | 910 | 1240 | 1690 |
| C | | | | | | | | | | | | |
| Drop/Gain in | | 70% | −1% | 17% | 28% | 15% | 6% | 0% | 6% | 7% | 46% | 140% |
| viscosity | | | | | | | | | | | | |
| Composition | 108 | 380 | 604 | 610 | 720 | 740 | 800 | 720 | 740 | 560 | 460 | 590 |
| D | | | | | | | | | | | | |
| Drop/Gain in | | 252% | 59% | 61% | 89% | 95% | 111% | 89% | 95% | 47% | 21% | 127% |
| viscosity | | | | | | | | | | | | |
| Composition | 4650 | 5410 | 4760 | 4500 | 4190 | 4080 | 4190 | 4240 | 4480 | 5280 | 5670 | 6640 |
| E | | | | | | | | | | | | |
| Drop/Gain in | | 16% | −12% | −17% | −23% | −25% | −23% | −22% | −17% | −2% | 5% | 37% |
| viscosity | | | | | | | | | | | | |

This table shows that a drop in viscosity is observed for the reference polymer (Composition A) and that it increases from 8 days to end with a loss of viscosity reaching 37% after 34 days.

For compositions B to E (according to the invention), the fall in viscosity observed over time is reduced and compensated for by the degradation of the labile cross-linker.

1. A polymeric composition for thickening a cosmetic or detergent composition comprising:
at least one polymer cross-linked with at least one labile cross-linker and at least one non-labile cross-linker; or
at least two cross-linked polymers, at least one being cross-linked with at least one labile cross-linker, and at least one being cross-linked with at least one non-labile cross-linker.

2. Polymer composition according to claim 1, wherein the cross-linked polymer is a synthetic polymer obtained from at least one monomer having ethylenic unsaturation.

3. Polymer composition according to claim 1, wherein the labile cross-linker is chosen from diamine diacrylamides and methacrylamides or methacrylate esters, and vinyl or allyl esters of di- or tri-functional acids.

4. Polymer composition according to claim 1, wherein the amount of labile cross-linker in the cross-linked polymer is between 100 and 200,000 ppm based on the total moles of monomer.

5. Polymer composition according to claim 1, wherein the non-labile cross-linker is chosen from methylene bisacrylamide, diallylamine, triallylamine, divinylsulphone and diethylene glycol diallyl ether.

6. Polymer composition according to claim 1, wherein the amount of non-labile cross-linker in the cross-linked polymer is between 1 and 2000 ppm based on the total moles of monomer.

7. Polymer composition according to claim 1, wherein the cross-linked polymer is a cationic or anionic polymer.

8. The composition according to claim 1, wherein it is an aqueous gel.

9. Cosmetic or detergent composition comprising at least one polymeric composition as described in claim 1.

10. Cosmetic or detergent composition according to claim 9, wherein it contains an amount of polymeric composition of between 0.01 and 5% by weight.

11. Use of at least one polymeric composition according to claim 1 to viscosify a cosmetic or detergent composition.

12. Use of at least one polymeric composition according to claim 1 for maintaining the viscosity of a cosmetic or detergent composition over time.
